(19) United States
(12) Patent Application Publication
(10) Pub. No.: US 2008/0132893 A1
(43) Pub. Date: Jun. 5, 2008

(54) Electrosurgical system

(75) Inventors: Frank D'Amelio, Los Olivos, MI (US); Andrew John Ford, Somerset (GB)

Correspondence Address: NIXON & VANDERHYE, PC, 901 NORTH GLEBE ROAD, 11TH FLOOR, ARLINGTON, VA, 22203, US

(73) Assignee: Gyrus Group plc, Berkshire (GB)

(21) Appl. No.: 11/979,844

(22) Filed: Nov. 8, 2007

Related U.S. Application Data
(60) Provisional application No. 60/857,483, filed on Nov. 8, 2006.

(51) Int. Cl. A61B 18/14

(52) U.S. Cl. 606/52

(57) ABSTRACT

An electrosurgical system includes an electrosurgical instrument and a user alert means (78). The electrosurgical instrument includes an elongate body (1) having a proximal end (2) and a distal end (3), and a pair of jaws (12) carried by the elongate body and disposed at the distal end. An actuator mechanism (5) at the proximal end of the elongate body (1) is movable by the user of the instrument to open and close the jaws (12). The instrument includes first and second electrodes (13), (14) carried by the jaws (12), and at least one strain gauge (75) adapted to provide a signal representative of the closure force applied by the jaws (12). The strain gauge (75) sends signals to the user alert means (78), the user alert means supplying the user of the instrument with an indication (82) of the closure force being applied by the instrument.

### CROSS-REFERENCE TO RELATED APPLICATION

This application claims the benefit of Provisional Application No. 60/857,483, filed Nov. 8, 2006, the entire content of which is hereby incorporated by reference in this application.

### BACKGROUND OF THE INVENTION

This invention relates to an electrosurgical system including a bipolar electrosurgical instrument for use in the cutting and sealing of tissue.

### BRIEF SUMMARY OF THE INVENTION

There are many examples of endoscopic forceps devices for the sealing and/or cutting of tissue, and the present invention attempts to provide an improved system for the cutting or sealing of tissue.

Accordingly, an electrosurgical system is provided including an electrosurgical instrument and a user alert means, the electrosurgical instrument including:

i) an elongate body having a proximal end and a distal end,

ii) a pair of jaws carried by the elongate body and disposed at the distal end thereof,

iii) an actuator mechanism carried by the elongate body at the proximal end thereof and movable by the user of the instrument to open and close the jaws,

iv) first and second electrodes carried by the jaws, and

v) at least one strain gauge adapted to provide a signal representative of the closure force applied by the jaws,

the arrangement being such that the at least one strain gauge sends signals to the user alert means, the user alert means supplying the user of the instrument with an indication of the closure force being applied by the instrument.

The importance of maintaining a proper closure force during electrosurgical sealing or cutting has already been appreciated, for example in U.S. Pat. Nos. 5,776,130, 6,179,834 and 6,039,733. Too high a closure force can result in the rupturing of the tissue, while too low a closure force can result in incomplete or inadequate sealing of the tissue. The above US patents describe instruments in which the closure force is maintained by means of a “lost motion connection”. The prior art does not provide user feedback to assist the user of the system in knowing whether the closure force being applied is within recommended limits. It is believed that the user alert means of the present invention supplies this missing assistance.

U.S. Pat. Nos. 6,743,229 and 6,726,686 state the desire for a closure force within certain limits, but provide this by way of a ratchet mechanism. U.S. Pat. No. 7,025,764 discloses the use of a strain gauge on a forceps device, but this is for the measurement of tissue thickness, not closure force. The present applicants have appreciated that a user alert as to closure force provides useful information to the user of the system.

According to a preferred arrangement, the user alert means is a display, adapted to display a visual representation of the closure force being applied by the instrument. The visual representation can be a numerical representation of the closure force, or more conveniently a graphical representation of the closure force being applied by the instrument. In one arrangement, the display is in the form of a bar graph, with different regions marked for different closure force. A first region can indicate too low a closure force, a second region can indicate the recommended range for the closure force, and a third region can indicate too high a closure force. To warn the user, the first and third regions can be indicated in red, with the second recommended region in green.

The system preferably includes an electrosurgical generator for supplying RF energy to the first and second electrodes, in which case the display means is conceivably mounted on the electrosurgical generator. More typically, where the system includes an endoscopic camera, and a monitor is provided to display an image captured by the endoscopic camera, the display means is constituted by the monitor, and the visual representation of the closure force being applied by the instrument is displayed in addition to the image captured by the endoscopic camera. In this way, the user of the system can see an image of the tissue being grasped by the jaws of the instrument, and also a graphical representation of whether the closure force being applied to the tissue is within recommended limits.

Alternatively or additionally, the user alert means is an audible signal representative of the closure force being applied by the instrument. For example, the audible signal could change in pitch depending on the closure force being applied to the tissue, or could change from an intermittent “bleep” to a constant tone when the closure force is within the prescribed range. An excess closure force could result in a different tone, such as a warning siren.

There are several locations in which the at least one strain gauge can be located on the instrument, in order to give an indication of the closure force being applied to the tissue. According to a first arrangement, the at least one strain gauge is located on one of the jaws of the instrument. Alternatively, the at least one strain gauge can be located on the actuator mechanism. Where the body of the instrument includes first and second elements, one being movable with respect to the other in order to effect the opening and closing of the jaws, the at least one strain gauge is conveniently located on one of the first and second elements. In this way, when the actuator mechanism is moved, and the first element moves relative to the second element in order to close the jaws, the strain gauge senses this movement to give an indication of the closure force being applied to the tissue.

### BRIEF DESCRIPTION OF THE DRAWINGS

The invention will now be described in more detail, by way of example only, with reference to the accompanying drawings, in which;

FIG. 1 is a schematic sectional view of an endoscopic electrosurgical instrument forming part of a system in accordance with the invention;

FIG. 2 is a perspective view of the jaw assembly of the instrument of FIG. 1;

FIG. 3 is a cross-sectional view of the body of the instrument of FIG. 1;

FIG. 4 is an end view of the jaw assembly of FIG. 2;

FIG. 5 is a side view of the jaw assembly of FIG. 2;

FIG. 6 is a circuit diagram of a switching circuit used in conjunction with the electrosurgical instrument of FIG. 1;

FIG. 7 is a schematic side view of an alternative embodiment of electrosurgical instrument for use in open procedures and forming part of a system in accordance with the invention;

FIG. 8 is an enlarged view of a portion of one of the jaws of the instrument of FIG. 7;

FIG. 9 is a sectional end view of one of the jaws of the instrument of FIG. 7;

FIG. 10 is a sectional end view of the other jaw of the instrument of FIG. 7;

FIG. 11 is a schematic drawing showing the possible location of strain gauges on the instrument of FIG. 1;

FIG. 12 is a schematic cross-sectional view of an endoscopic electrosurgical instrument forming part of a system in accordance with the invention;

FIG. 12A is an enlarged cross-sectional view of a part of FIG. 12;

FIG. 13 is a schematic cross-sectional view of an alternative embodiment of endoscopic electrosurgical instrument forming part of a system in accordance with the invention;

FIGS. 13A and 13B are enlarged cross-sectional views of parts of FIG. 13;

FIG. 14 is a schematic representation of an electrosurgical system in accordance with the invention; and

FIG. 15 is a circuit diagram of a circuit used in conjunction with an alternative embodiment of electrosurgical system in accordance with the invention.

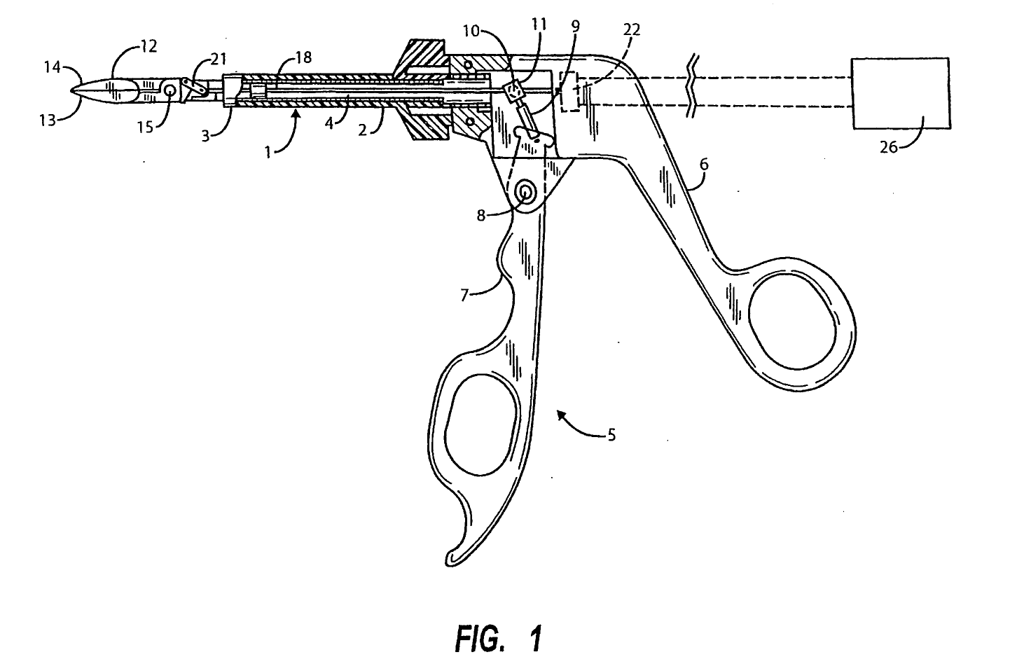

Referring to FIG. 1, a bipolar forceps device includes an elongated tubular shaft 1 with a proximal end 2, a distal end 3, and a lumen 4 which extends for the entire length of the tubular member. At the proximal end 2 of the tubular member 1 is a scissors-type handle assembly 5 with a first handle 6 and a second handle 7. The second handle 7 is pivotable with respect to the first handle 6, about a pivot pin 8. In a known design of actuation mechanism, the second handle 7 has a pin 9 affixed to the top thereof, such that movement of that handle causes a corresponding movement to a sphere 10 supported in a U-shaped cradle 11.

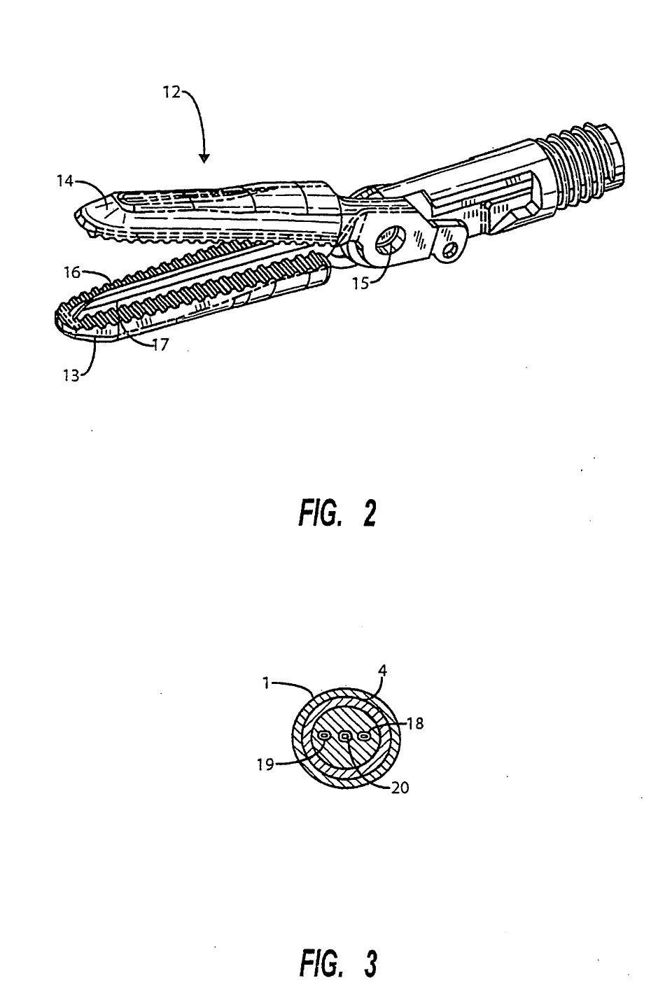

Fitted into the distal end 3 of the tubular member 1 is a forceps jaw assembly 12, more particularly shown in FIG. 2. The jaw assembly 12 comprises a first jaw member 13 and a second jaw member 14, pivotally joined to each other by an insulated rivet 15. The jaw member 13 is provided with a relatively-long, but narrow cutting electrode 16 isolated there from by a ceramic insulator 17. As shown in FIG. 3, three rigid electrically-conductive rods 18, 19 and 20, each covered with a layer of electrical insulation, extend through the lumen 4 of the tubular member 1. The rods 18, 19 are pivotally connected to the respective jaw members 13, 14 by rigid links 21, whilst the rod 20 is connected by means of a wire 24 (as best shown in FIG. 5) to the cutting electrode 16. The proximal ends of the rods 18, 19 and 20 extend from the tubular member 1 through the sphere 10 and terminate in a connector 22, by which means the device can be attached to an electrosurgical generator 26.

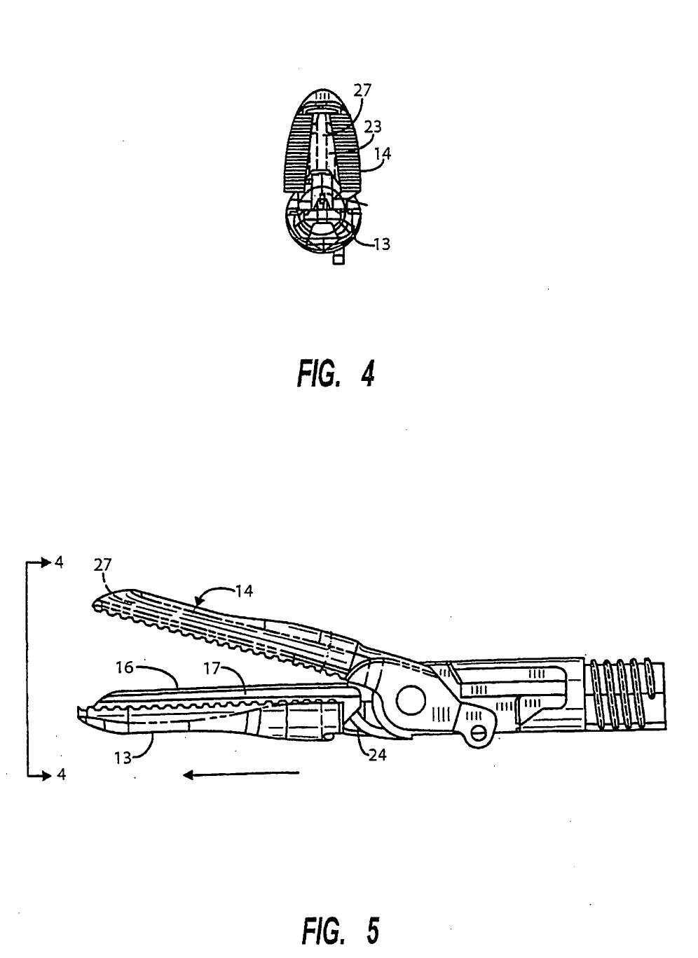

As shown in FIG. 2, the cutting electrode 16 is in the form of an elongate rail, extending along the length of the jaw member 13. The rail 16 is mounted on top of the ceramic insulator 17 such that it is insulated from the conductive jaw member 13. The rail 16 is typically 50 to 100 microns in width, and protrudes from the ceramic insulator 17 by a distance of approximately 50 microns. When the jaw assembly 12 is in its closed position, the rail 16 is received in a corresponding longitudinal recess 23 in the jaw member 14, best shown in FIG. 4. A compressible strip 27 of insulting material is provided in the recess 23. The device can be used to coagulate tissue, using the jaw assembly 12 in its closed position. The jaw assembly 12 is closed, capturing tissue between the jaw member 13 and the jaw member 14. The cutting rail 16 is received in the recess 23 and, without the supply of an electrosurgical cutting signal thereto, does not have a cutting effect on the tissue there between. A coagulating signal from the electrosurgical generator 26 is supplied between the jaw members 13 and 14, via the rods 18 and 19. This causes the coagulation of the tissue held between the jaws 13 and 14.

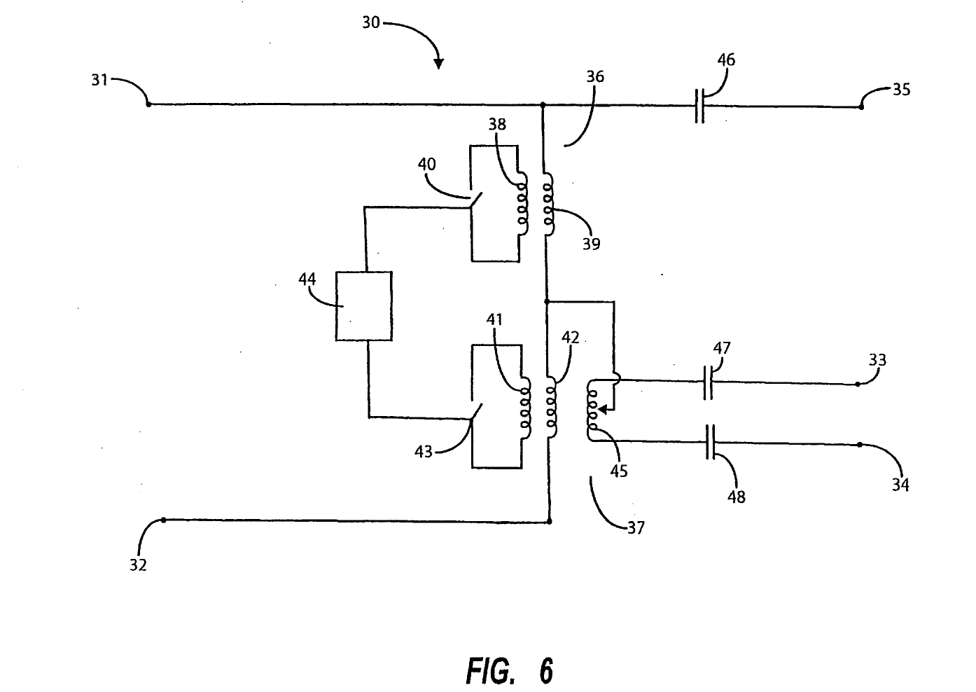

The device can also be used in a blended cutting and coagulation mode, as described in our U.S. Pat. No. 6,966,907. An example of an electrical circuit to provide such an arrangement is shown in FIG. 6. The circuit is shown generally at 30 and may be provided as a part of the output stage of the generator 26, as a part of the forceps instrument, or as a separate unit located between the generator and the instrument. Whichever arrangement is employed, input connections 31 and 32 are connected to the output of the generator 26, and output connections 33 and 34 are connected to the rods 18 and 19, and hence to the jaw members 13 and 14. An output connection 35 is connected to the rod 20, and hence to the cutting electrode 16.

Between the input connections 31 and 32 there is a bridge circuit comprising a first transformer 36 and a second transformer 37. The first transformer 36 comprises a primary winding 38 and a secondary winding 39. A switch element 40 is provided in parallel with the primary winding 38. The second transformer 37 comprises a primary winding 41 and a secondary winding 42. A switch element 43 is provided in parallel with the primary winding 41. The switch elements 40 and 43 are operated by a control unit 44.

The second transformer 37 is a step-down transformer in which the secondary winding 42 is itself the primary to a further centre-tapped secondary winding 45 connected across the output connections 33 and 34. An isolation capacitor 46 is provided between the bridge circuit and the output connection 35, and isolation capacitors 47 and 48 are provided between the bridge circuit and the output connections 33 and 34.

The operation of the circuit is as follows. For a predetermined period, the control unit 44 operates the switch 43 to close and provide a short circuit across the primary winding 41 of the second transformer 37. In this arrangement, with the secondary transformer 37 effectively short-circuited, the output of the generator 26 is directed between the output connection 35 and both of the output connections 33 and 34. This has the effect of energizing the cutting rail 16 with a cutting voltage, as compared to the jaw members 13 and 14, which effectively act as return electrodes for the electrosurgical cutting operation.

After a predetermined period, the control unit 44 operates to open the switch 43 and then close the switch 40 to provide a short circuit across the primary winding 38 of the first transformer 36. There is a short predetermined delay between the opening of the switch 43 and the closing of the switch 40 to ensure that both switches are never closed at the same time (as this would provide a short circuit across the output connections of the generator 26). With the switch 40 closed, the first transformer 36 is effectively short-circuited, and the output of the generator 26 is directed entirely to the second transformer 37. The second transformer 37 is a step-down transformer, and provides a lower voltage signal between the output connections 33 and 34. This has the effect of energizing the first and second jaw members 13 and 14 with a coagulating voltage.

After a predetermined time, the control unit 44 opens the switch 40 and then closes the switch 43, reverting to the arrangement initially described in which a cutting voltage is delivered to the cutting rail 16. By constantly alternating between the two conditions herein described, the circuit provides a rapidly alternating cut and coagulation signal to a forceps device connected thereto. In this way, the forceps device is able to cut tissue, while simultaneously coagulating the tissue in order to curtail bleeding.

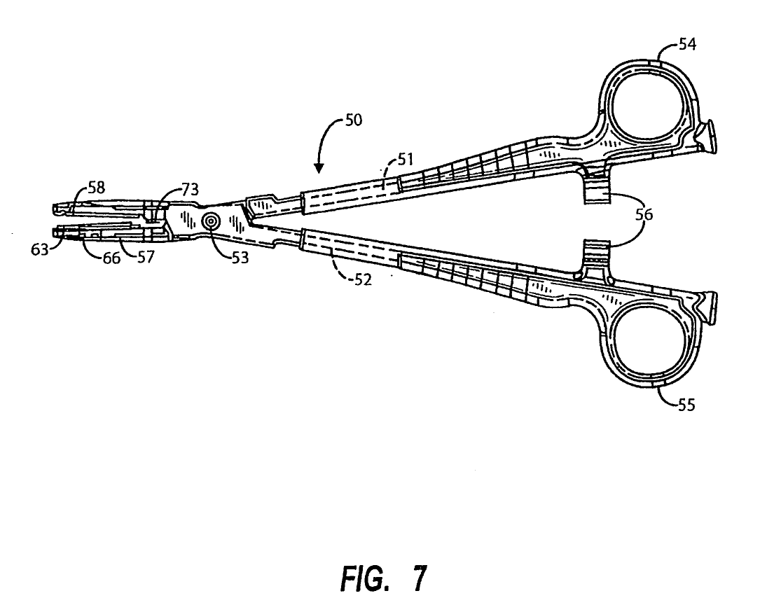

Although the forceps device of FIGS. 1 to 5 is shown as an endoscopic instrument, the invention can also be employed in connection with open instruments, as will be described with reference to FIGS. 7 to 10. The instrument shown generally at 50, comprises two longitudinal members 51 and 52, mounted for pivotal movement by means of a pivot pin 53. The proximal end of the member 51 is in the form of handle portion 54, and the proximal end of the member 52 is in the form of a handle portion 55. A ratchet mechanism 56 is provided on each handle portion 54, 55 for locking the handle portions when they are moved together into their closed position.

Distal of the pivot pin 53, the longitudinal member 51 forms a jaw member 57, while the longitudinal member 52 forms a jaw member 58. Movement of the handle portions 54 and 55 causes the jaw members 57 and 58 to open and close.

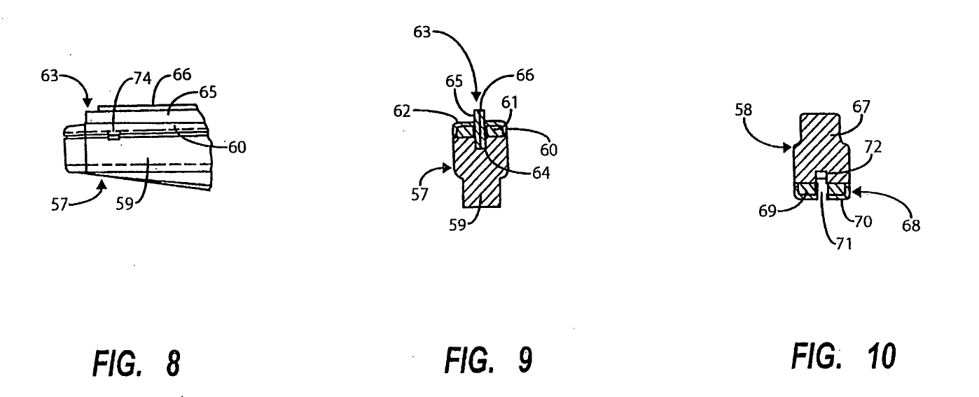

With reference to FIGS. 8 and 9, the jaw member 57 comprises an integral base portion 59 on which is mounted a shim member 60, secured by means of clips 74. The shim member 60 comprises an insulating strip 61 covered by a metallic surface electrode 62. A cutting electrode assembly 63 is mounted in a recess 64 running longitudinally along the jaw member 57. The cutting electrode assembly 63 comprises a raised insulator block 65, typically of a ceramic material, and a cutting electrode 66 mounted in a further longitudinal recess in the insulator block. The cutting electrode 66 is typically 100 microns in width, and protrudes from the insulator block 65 by a distance of approx 425 microns.

The opposite jaw member 58 (shown in FIG. 10) also comprises a base portion 67 and a shim member 68. The shim member 68 also comprises an insulting strip 69 covered by a metallic surface electrode 70. The shim member 68 includes a central recess 71 in which the cutting electrode assembly 63 of the jaw member 57 can be received when the jaw members 57 and 58 are in their closed position. At the base of the recess 71 is a strip 72 of resilient material such as an elastomer, such that the cutting electrode 66 bears against that strip when the jaw members 57 and 58 are closed one against the other. A stop member 73, mounted on one of the jaws, regulates the separation of the jaws when they are in their closed position.

The operation of the instrument 50 will now be described. The jaw members 57 and 58 are moved to their closed position, gripping tissue to be cut there between. Then a first coagulating RF signal is supplied between the surface electrodes 62 and 70, causing the coagulation of the tissue held between the jaw members 57 and 58. Without releasing the tissue, a second cutting RF signal is then supplied to the cutting electrode 66, causing the cutting of the tissue held by the jaw members 57 and 58.

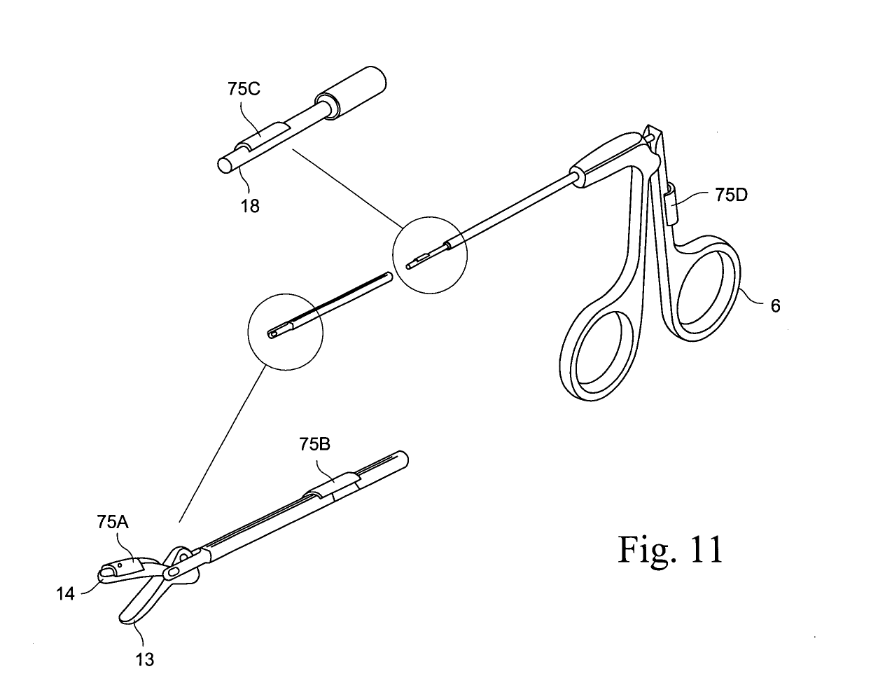

Whether the instrument is an endoscopic instrument as shown in FIGS. 1 to 5, or an open instrument as shown in FIGS. 7 to 10, the closure force applied to the tissue by the user of the instrument is important, especially for ensuring the adequate sealing or coagulation of the tissue. FIG. 11 shows an instrument in which strain gauges 75 are located on the instrument. Strain gauge 75A is located on the jaw 14, while strain gauge 75B on the outside of the tubular shaft 1. Strain gauge 75C is located on the push rod 18, while strain gauge 75D is located on the handle 6. Clearly, it is not necessary to have strain gauges at all of these locations, and only a single strain gauge may be provided, but the above illustrates several possible locations for the gauges.

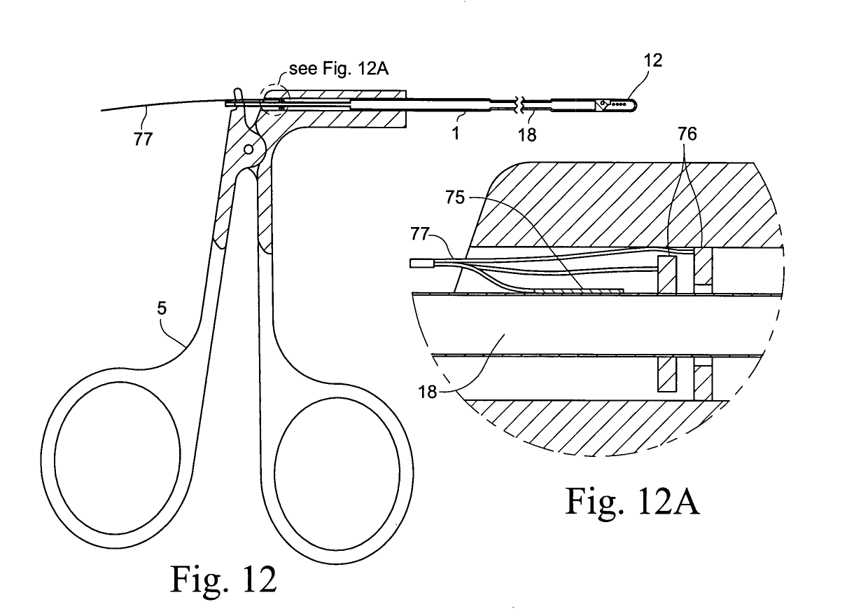

FIGS. 12 and 12A show in more detail an alternative location for the strain gauge 75. The strain gauge is affixed to the push rod 18 at the proximal end thereof, and there is also provided a linear distance gauge 76 to determine the extent of the longitudinal movement of the push rod. Signals from the strain gauge 75 and the linear distance gauge 76 are transmitted via leads 77 to a display unit (not shown in FIGS. 12 and 12A).

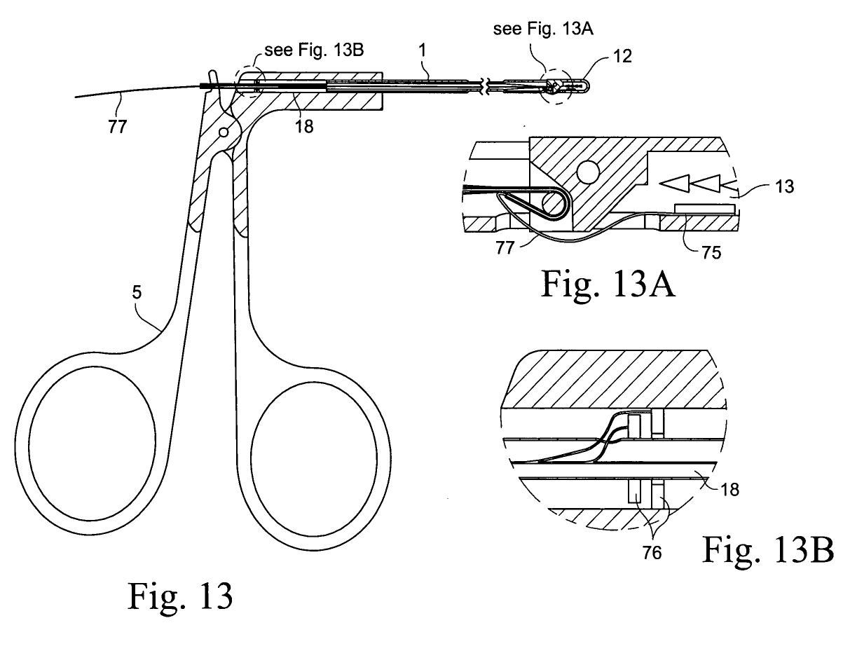

FIGS. 13, 13A and 13B show another alternative arrangement for the strain gauge 75. The strain gauge 75 is located on jaw 13 and signals there from are transmitted via lead 77 to a display unit (not shown in FIGS. 13, 13A and 13B). The linear distance gauge 76 is located as before at the proximal end of the push rod 18.

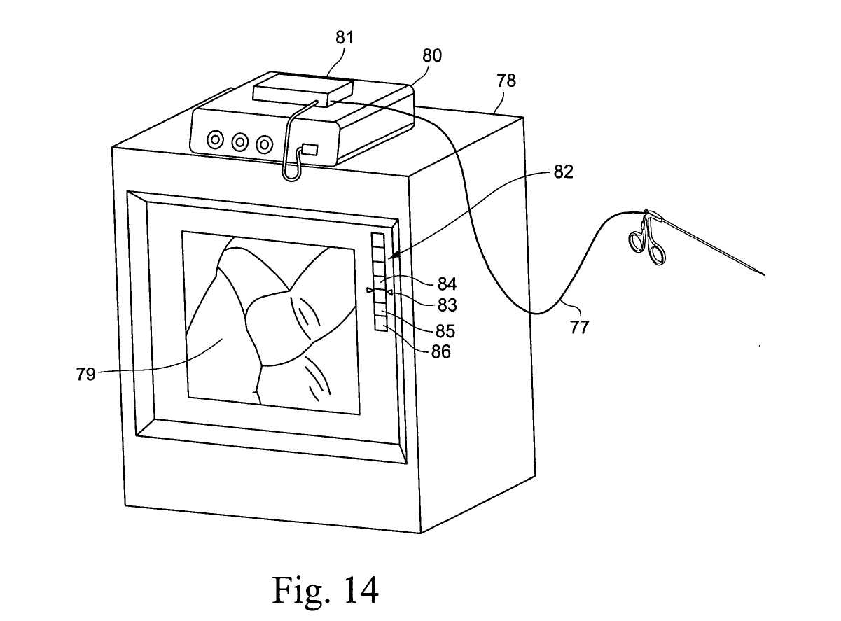

FIG. 14 shows the display unit on which the signals from the strain gauges 75 are displayed. The display unit is in the form of a monitor 78 showing an image 79 of the surgical site captured by an endoscopic camera (not shown). The camera sends signals to a control unit 80, which processes the signals and produces the image 79 on the monitor 78, in conventional fashion. Signals from the strain gauges 75 are transmitted via lead 77 to a signal controller 81, attached to the control unit 80. The signal controller produces a graphical display 82 from the strain gauge signals, and superimposes this graphical display on the image 79. The electrosurgical generator 26 (not shown in FIG. 14) supplies RF energy to the instrument.

The graphical display 82 is a bar chart, with regions of different colours to demonstrate optimum, sub-optimum, and non-recommended levels of closure force. A floating pointer 83 moves up and down between the regions to show the current closure force being exerted by the user of the instrument. The user attempts to maintain the closure force such that the pointer 83 is in the central green-coloured region 84, as opposed to the intermediate yellow-coloured regions 85, or the outermost red-coloured region 86. This provides an easy to interpret, visual guidance as to the closure force exerted by the user,

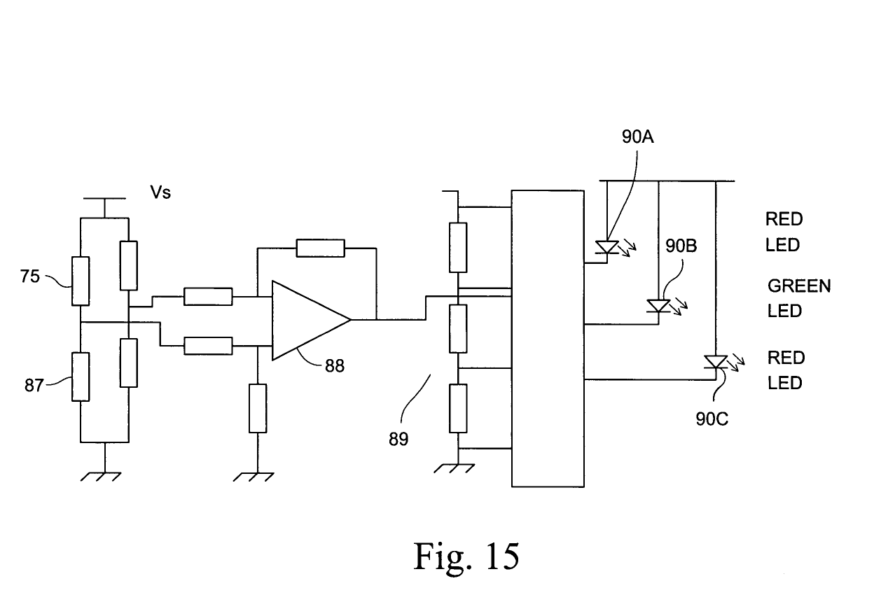

FIG. 15 shows a circuit diagram for an alternative arrangement, in which one or more reference strain gauges 87 are provided in addition to the strain gauges 75. The reference strain gauges 87 are typically provided at 90 degrees to the strain gauges 75, in order to cancel out any strain produced by changes in temperature etc. The signals from the strain gauges 75 and 87 are amplified by means of an amplifier circuit 88, and compared by means of a comparator circuit 89. The comparator circuit lights one of a series of LEDs 90 depending on whether the compared signals are too high, too low, or within a preferred range. A red LED 90A is illuminated if the compared signals are above a first prescribed threshold, indicating that the closure force being applied is too high. Conversely, a red LED 90C is illuminated if the compared signals are below a second prescribed threshold, indicating that the closure force being applied is too low. Finally, a green LED 90B is illuminated if the compared signals are between the first and second prescribed thresholds, indicating that the closure force being applied is within the preferred range.

Instead of illuminating LEDs 90, the output from the comparator circuit may be used to display a numerical value for the closure force, or to generate an audible signal to guide the user as to whether there is a need to increase or decrease the closure force being applied to the tissue. In this way, the incidence of the incomplete or inadequate sealing of tissue is reduced. In addition, with the provision of a visual or audible indication of closure force, it may be possible to dispense with the ratchet mechanism 56 for the open forceps shown in FIGS. 7 to 10. This may make the instrument easier and quicker to use, whether it be for coagulation or the cutting of tissue.

This invention has been described herein in considerable detail in order to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention.

While the invention has been described in connection with what is presently considered the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. An electrosurgical system including an electrosurgical instrument and a user alert means, the electrosurgical instrument including:
i) an elongate body having a proximal end and a distal end,
ii) a pair of jaws carried by the elongate body and disposed at the distal end thereof,
iii) an actuator mechanism carried by the elongate body at the proximal end thereof and movable by the user of the instrument to open and close the jaws,
iv) first and second electrodes carried by the jaws, and
v) at least one strain gauge adapted to provide a signal representative of the closure force applied by the jaws,
the arrangement being such that the at least one strain gauge sends signals to the user alert means, the user alert means supplying the user of the instrument with an indication of the closure force being applied by the instrument.

2. An electrosurgical system according to claim 1 wherein the user alert means is a display, adapted to display a visual representation of the closure force being applied by the instrument.

3. An electrosurgical system according to claim 2 wherein the display is adapted to display a graphical representation of the closure force being applied by the instrument.

4. An electrosurgical system according to claim 1 wherein the system includes an electrosurgical generator for supplying RF energy to the first and second electrodes.

5. An electrosurgical system according to claim 2 wherein the display means is mounted on the electrosurgical generator.

6. An electrosurgical system according to claim 1 wherein the system includes an endoscopic camera, and a monitor adapted to display an image captured by the endoscopic camera.

7. An electrosurgical system according to claim 2 wherein the display means is constituted by the monitor, and the visual representation of the closure force being applied by the instrument is displayed in addition to the image captured by the endoscopic camera.

8. An electrosurgical system according to claim 1 wherein the user alert means is an audible signal representative of the closure force being applied by the instrument.

9. An electrosurgical system according to claim 1 wherein the at least one strain gauge is located on one of the jaws of the instrument.

10. An electrosurgical system according to claim 1 wherein the at least one strain gauge is located on the actuator mechanism.

11. An electrosurgical system according to claim 1 wherein the body of the instrument includes first and second elements, one being movable with respect to the other in order to effect the opening and closing of the jaws.

12. An electrosurgical system according to claim 11 wherein the at least one strain gauge is located on one of the first and second elements.

13. An electrosurgical system including an electrosurgical instrument and a user alert means, the electrosurgical instrument including:
i) an elongate body having a proximal end and a distal end,
ii) a pair of jaws carried by the elongate body and disposed at the distal end thereof,
iii) an actuator mechanism carried by the elongate body at the proximal end thereof and movable by the user of the instrument to open and close the jaws,
iv) first and second electrodes carried by the jaws, and
v) at least one force sensing device associated with an element of the instrument operable to transmit a jaws closure force, the sensing device being adapted to provide an electrical signal representative of the closure force,
the arrangement being such that the at least one force sensing device sends signals to the user alert means, the user alert means supplying the user of the instrument with an indication of the closure force being applied by the instrument.
